(19) United States
(12) Patent Application Publication
(10) Pub. No.: US 2023/0263612 A1
(43) Pub. Date: Aug. 24, 2023

(54) TISSUE EXPANDER WITH EXTENDED INFLATION MECHANISM USING CONTROLLED CHEMICAL REACTION

(71) Applicant: VIOLOGEN, INC., Fremont, CA (US)

(72) Inventor: Sung-Mi LEE, Seoul (KR)

(21) Appl. No.: 17/678,558

(22) Filed: Feb. 23, 2022

(51) Int. Cl. A61F 2/00

(52) U.S. Cl. CPC A61F 2/0059; A61F 2210/0061; A61F 2210/0076; A61F 2230/0076; A61F 2250/0003

(57) ABSTRACT

Embodiments of the present disclosure relate to a tissue expander that expands in a gradual manner using a controlled release of gas resulting from a chemical reaction of a reactant substance within the tissue expander. The reactant substance is initially in a liquid or solid state and is stored in an internal chamber of the tissue expander. The chemical reaction of the reactant substance produces the gas that causes the tissue expander to expand over time. The tissue expander continues to expand gradually after implanting of the tissue expander within a patient’s body and initiating of the chemical reaction.

### BACKGROUND

### 1. Field of the Disclosure

The present disclosure relates to a tissue expander, more specifically to a tissue expander that self-inflates using controlled chemical reaction.

### 2. Description of the Related Arts

A reconstructive surgery is generally performed to replace a patient’s damaged tissue with normal tissue. Such reconstructive surgery is often employed, for example, when a part of the tissue is damaged, when a scar stretches across a large area, when a malignant tumor is removed or when the patient suffers a significant injury. The reconstructive surgery has seen a high demand to treat or recover from, among others, burn scars, vascular anomalies, large scars, large nevi, and breast reconstruction.

A tissue expander is used in such reconstructive surgery. The tissue expander is inserted into a patient’s surgical site and is then expanded to stretch the patient’s tissue as part of a reconstructive surgery. However, an abrupt expansion of the tissue expander tends to cause a significant pain and suffering to the patient. Moreover, the tissue expander is often expanded multiple times over a prolonged time. For each expansion process, the patient would visit a medical facility, which tends to increase the overall cost of the reconstructive surgery as well as cause inconvenience to the patient. Moreover, abrupt inflation of the tissue expander may rupture the suture at the patient’s surgical site and/or increase its weight that causes tissue around the surgical site to sag.

### SUMMARY

Embodiments relate to tissue expander with a flexible enclosure at least part of which is configured to contact and expand a patent’s surgical site by a chemical reaction that continues to occur after implanting the flexible enclosure into the patent’s surgical site. The flexible enclosure has an internal chamber and is impermeable to gas. The internal chamber contains a first reactant substance as liquid or solid material and produces the gas as a result of the chemical reaction of the first reactant substance. The produced gas inflates the flexible enclosure.

In one or more embodiments, the flexible enclosure further comprises a second reactant substance that causes the chemical reaction after the second reactant substance comes into contact with the first reactant substance.

In one or more embodiments, the first reactant substance comprises a gas producing metal or sodium percarbonate, and the second reactant substance comprises aqueous solution.

In one or more embodiments, the gas producing metal is a metal selected from a group consisting of magnesium, iron, molybdenum, tungsten and zinc.

In one or more embodiments, the flexible enclosure is formed with a storage chamber that encloses the second reactant substance, a passage between the internal chamber and the storage chamber, and a collapsible wall in the passage. The collapsible wall is ruptured by an external force to enable the second reactant substance to flow into the internal chamber.

In one or more embodiments, the flexible enclosure is formed with at least one additional storage chamber that encloses the second reactant substance, and additional passages between the internal chamber and the additional storage chambers, and additional walls configured to be ruptured by additional external force to provide additional second reactant substance to the internal chamber.

In one or more embodiments, the first reactive substance is coated with a coating material to expedite, delay or prolong the chemical reaction.

In one or more embodiments, the internal chamber is filled with second reactant substance and the first reactant substance is enclosed in a casing that prevents the chemical reaction. The encapsulation is ruptured to expose the first reactant substance to the second reactant substance to cause the chemical reaction.

In one or more embodiments, the tissue expander further includes a septum attached to the flexible enclosure. The septum is penetrated to provide second reactant substance into the internal chamber that causes the chemical reaction. The septum is sealed after providing the second reactant substance into the internal chamber.

In one or more embodiments, the tissue expander includes a flexible conduit extending into the internal chamber to carry second reactance substance into the internal chamber to cause the chemical reaction.

In one or more embodiments, the tissue expander further includes a valve assembly inside the flexible enclosure or the valve assembly is connected to the flexible enclosure. The valve assembly injects second reactant substance that causes the chemical reaction after the second reactant substance comes into contact with the first reactant substance.

In one or more embodiments, the valve assembly includes a wireless communication circuit and a valve control circuit coupled to the communication circuit. The wireless communication circuit receives a wireless signal that instructs the valve control circuit to inject the second reactant substance.

In one or more embodiments, the tissue expander includes an electrolysis device in the internal chamber. The electrolysis device has internal space for storing the first reactive substance, and includes electrodes that perform electrolysis of the first reactant substance to produce the gas, and a control circuit connected to the electrodes to provide current to the electrodes to perform the electrolysis.

In one or more embodiment, the tissue expander further includes a wireless communication circuit that receives a wireless signal instructing the control circuit to provide the current to the electrodes.

### BRIEF DESCRIPTION OF THE DRAWINGS

FIG. 1 is a diagram illustrating a tissue expander implanted at a patient’s surgical site, according to one embodiment.

FIG. 2A is a plan view of a tissue expander having an internal chamber and a storage chamber, according to one embodiment.

FIG. 2B is a cross-sectional diagram of the tissue expander of FIG. 2A, according to one embodiment.

FIG. 2C is a cross-sectional diagram of the tissue expander of FIG. 2A in an inflated state, according to one embodiment.

FIG. 2D is a cross-sectional diagram of a reactant substance coated with a coating material, according to one embodiment.

FIG. 3A is a plan view of a tissue expander having an internal chamber and a plurality of storage chambers, according to one embodiment.

FIG. 3B is a plan view of a tissue expander having different portions of reactant substance with varying coating thicknesses, according to one embodiment.

FIG. 3C is a graph illustrating the release of gas with sequential chemical reaction of the reactant substance, according to one embodiment.

FIG. 4A is a conceptual diagram illustrating a film of a tissue expander including four layers of materials, according to one embodiment.

FIG. 4B is a conceptual diagram illustrating a film of a tissue expander including five layers of materials, according to one embodiment.

FIGS. 5A and 5B are diagrams of tissue expander with reactant substance enclosed in an ampoule, according to one embodiment.

FIG. 6A is a conceptual diagram illustrating a tissue expander with a septum, according to one embodiment.

FIG. 6B is a conceptual diagram illustrating a tissue expander with a hose, according to one embodiment.

FIG. 7A is a perspective view of a tissue expander with a remotely controlled valve assembly, according to one embodiment.

FIG. 7B is a cross-sectional diagram of the valve assembly of FIG. 7A, according to one embodiment.

FIG. 7C is a perspective view of a tissue expander attached with a remotely controlled valve assembly, according to one embodiment.

FIG. 8 is a conceptual diagram of a tissue expander with an electrolysis device, according to one embodiment.

FIG. 9 is a flowchart illustrating a process of operating a tissue expander, according to one embodiment.

The figures depict, and the detail description describes, various non-limiting embodiments for purposes of illustration only.

### DETAILED DESCRIPTION

Reference will now be made in detail to embodiments, examples of which are illustrated in the accompanying drawings. In the following detailed description, numerous specific details are set forth in order to provide a thorough understanding of the various described embodiments. However, the described embodiments may be practiced without these specific details. In other instances, well-known methods, procedures, components, circuits, and networks have not been described in detail so as not to unnecessarily obscure aspects of the embodiments.

Embodiments of the present disclosure relate to a tissue expander that expands in a gradual manner using a controlled release of gas resulting from a chemical reaction of a reactant substance within the tissue expander. The reactant substance is initially in a liquid or solid state and is stored in an internal chamber of the tissue expander. The chemical reaction of the reactant substance produces the gas that causes the tissue expander to expand over time. The tissue expander continues to expand gradually after implanting of the tissue expander within a patient’s body after initiating the chemical reaction. Because the tissue expander inflates gradually over time, the patient experiences less pain and is subject to fewer visits to a medical facility for a reconstructive surgery. Further, the tissue expander does not increase its weight as a result of inflation, and hence, sagging or deformation of a surgical site may be avoided.

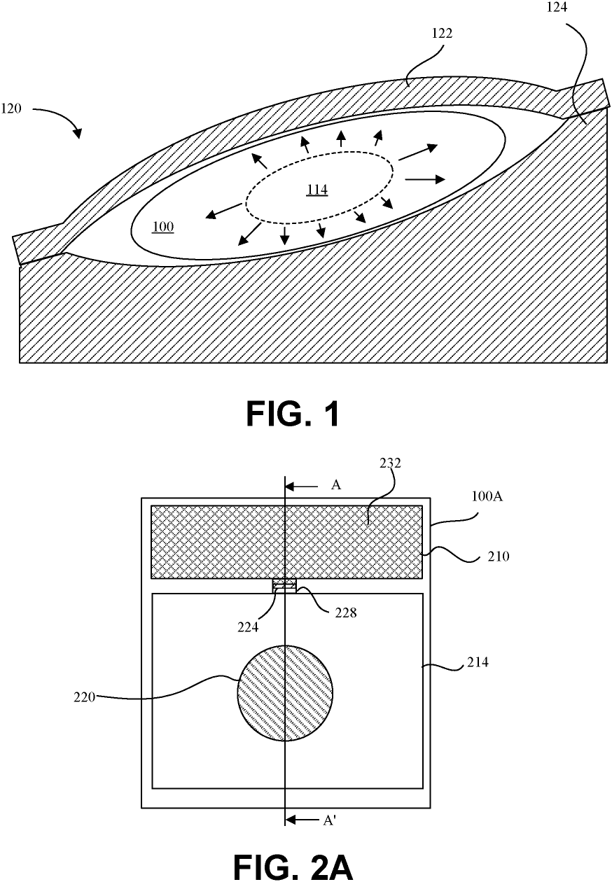

FIG. 1 is a diagram illustrating a tissue expander 100 implanted inside a patient’s surgical site, according to one embodiment. The tissue expander 100 is implanted in the surgical site and is typically placed below upper tissue 122 and above lower tissue 124. The upper tissue 122 is a portion of the tissue that is sought to be expanded by the tissue expander 100 while the lower tissue 124 may be subcutaneous tissue. As a result of chemical reaction 114 that occurs in the interior of the tissue expander 100, gas (shown by arrows) is produced within the tissue expander 100. The gas then exerts pressure on the interior walls of the tissue expander 100 so that the tissue expander 100 is inflated. As described below with reference to following figures, various mechanisms may be employed to produce the gas.

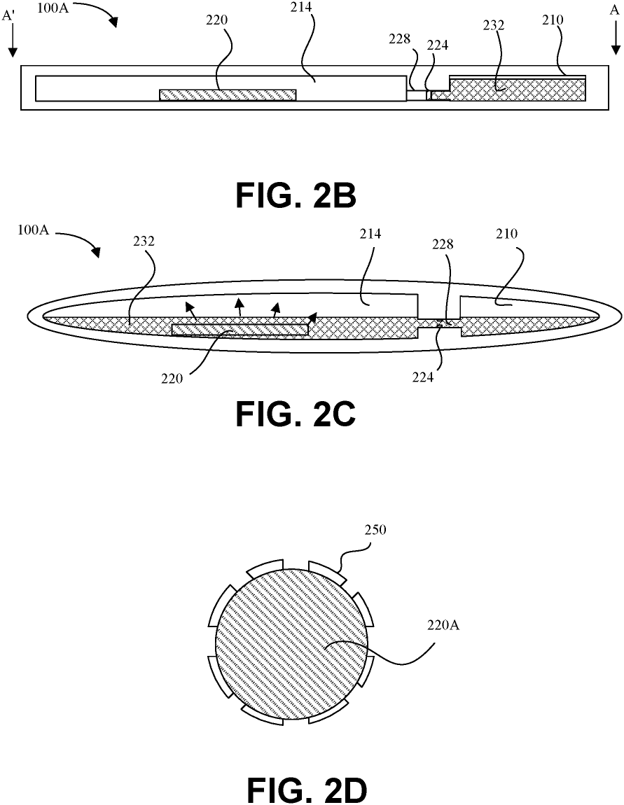

FIG. 2A is a plan view of a tissue expander 100A having an internal chamber 214 and a storage chamber 210, according to one embodiment. FIG. 2B is a cross-sectional diagram of the tissue expander 100A taken along line A-A′ of FIG. 2A. The internal chamber 214 includes first reactant substance 220 while the storage chamber 210 includes second reactant substance 232. The internal chamber 214 and the storage chamber 210 connected via a passage 228, but the passage 228 is initially blocked by a wall 224 that prevents the first and second reactant substances from coming into contact.

FIG. 2C is a cross-sectional diagram of the tissue expander 100A in an inflated state, according to one embodiment. Before, during or after implanting the tissue expander 100A in the patient’s surgical site (e.g., between the upper tissue 122 and the lower tissue 124), the wall 224 is ruptured. The rupturing of the wall 224 may be accomplished by exerting external force on the wall 224, for example, by squeezing the wall 224 by a surgeon’s fingers or by using a specialized tool to crack the wall 224. By rupturing the wall 224, the second reactant substance 232 may flow through the passage 228 into the internal chamber 214, and contact the first reactant substance 220. For this purpose, the second reactant substance 232 may be a liquid.

The first reactant substance 220 may be a solid material that produces gas upon contact with the second reactant substance 232. The first reactant substance comprises a gas producing metal or sodium percarbonate while the second reactant substance comprises aqueous solution (e.g., water or saline solution). The gas producing metal may include one or more of magnesium, iron, molybdenum, tungsten and zinc.

Taking an example of using magnesium as the first reactant substance 220 and water or saline solution as the second reactant substance 232, the chemical reaction can be expressed as follows:

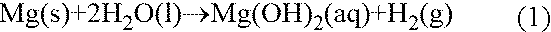

That is, when magnesium contacts water or saline solution, hydrogen gas is produced. 1 gram of magnesium undersoing such chemical reaction would produce 933 mL of hydrogen gas. Hence, by adjusting the amount of magnesium contained in the internal chamber 214, the final volume of the expanded tissue expander 100A can be estimated. Further, the chemical reaction is spontaenous yet not abrupt, and therefore, the amount of gas produced does not result in a sudden inflation of the tissue expander 100A.

In another example, sodium percarbonate is used as the first reactant substance 220 and water or saline solution is used as the second reactant substance 232. In this example, the chemical reaction can be expressed as follows:

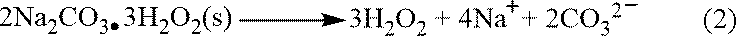

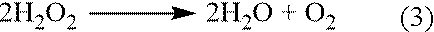

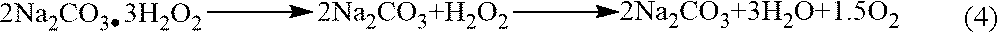

Similar to the example where magnesium is used, the amount of gas to be produced in this example may be controlled by adjusting the amount of sodium percarbonate or water (or the saline solution). According to the above chemical reaction, 2 moles of sodium percarbonate produces 1.5 moles of oxygen. For example, 1 gram of sodium percarbonate dissolving in water would produce 108 cc of oxygen gas.

FIG. 2D is a cross-sectional diagram of reactant substance 220A coated with a coating material 250, according to one embodiment. The coating material 250 has a different reactivity to the second reactant substance 232 so that the reaction rate can be adjusted. In one or more embodiments, the coating material 250 may increase the rate of the chemical reaction. Coating material 250 suitable for this purpose may include metal such as titanium, tantalum, gold or silver when magnesium is used, for example, as the reactant substance 220A. The coating material 250 may be porous or be partly coated on the reactant substance 220A to finely control the rate of the chemical reaction.

Conversely, the coating material 250 may decrease the rate of the chemical reaction. For this purpose, the coating material 250 may include polymer such as gelatin, polylactic acid (PLA), poly(lactic-co-glycolic acid) (PLGA) or ceramic material such as MgF₂ or hydroxyapatite (HA). By coating the first reactant substance 220A with polymer or ceramic material, the chemical reaction may be delayed or prolonged, and abrupt inflation of the tissue expander may be avoided.

Although FIGS. 2A through 2D illustrate only a single lump of the first reactant substance 220, 220A, multiple lumps of first reactant substance 220, 220A may be included in the internal chamber 214. Alternatively, the first reactant substance 220, 220A may be provided in powder form to increase the rate of chemical reaction. Further, although the shape of the lump of the first reactant substance 220, 220A is illustrated as being coin-shaped in FIGS. 2A through 2D, various other shapes may be employed to control the chemical reaction rate.

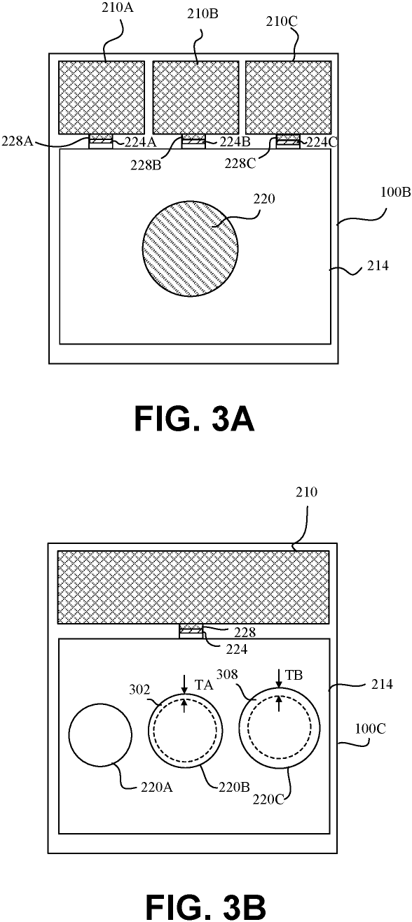

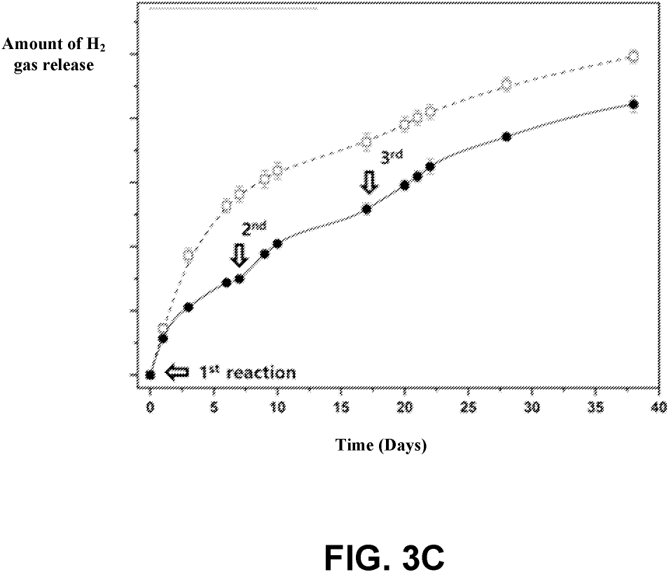

FIG. 3A is a plan view of tissue expander 100B having internal chamber 214 and a plurality of storage chambers 210A, 210B, 210C, according to one embodiment. The embodiment of FIG. 3A is different from the embodiment of FIGS. 2A through 2C in that a plurality of storage chambers 210A, 210B, 210C are provided to sequentially provide second reactant substance from the storage chambers 210A, 210B, 210C to the internal chamber 214. In this way, the chemical reaction between the first reactant substance 220 and the second reactant substance may occur sequentially in a more gradual and controlled manner.

Specifically, the tissue expander 100B is formed with passages 228A, 228B, 228C that are initially blocked by walls 224A, 224B, 224C. The walls 224A, 224B, 224C may be ruptured in sequence to inject the second reactant substance in the storage chambers 210A, 210B, 210C into the internal chamber 214. The rupturing of the walls 224A, 224B, 224C may be performed by the patient or a medical professional using fingers or a dedicated tool.

FIG. 3B is a plan view of tissue expander 100C having different portions 220A, 220B, 220C of first reactant substance with varying coating thicknesses, according to one embodiment. In the embodiment of FIG. 3B, instead of including multiple storage chambers, only a single storage chamber 210 is provided in the tissue expander 100C. However, different portions 220A, 220B, 220C are coated with coating materials that dissolve gradually overtime upon contact with the second reactant substance after passage 228 is opened by rupturing wall 224.

The first portion 220A of the first reactant substance may not include any coating or include only a thin layer of coating that dissolves quickly when exposed to the second reactant substance. The second portion 220B of the first reactant substance is coated with thickness TA and the third portion 220C of the first reactant substance is coated with thickness TB that is thicker than TA. Because of the absence or thin coating of the first portion 220A, the first portion 220A may start the chemical reaction immediately or shortly after the internal chamber 214 is injected with the second reactant substance. On the other hand, the second portion 220B and the third portion 220C of the first reactant substance start their chemical reaction after a longer time sufficient to dissolve the coating on the second and third portions 220B, 220C.

The material for coating different portions of the first reactant substance may include polymer such as gelatin, PLA, PLGA or ceramic material such as MgF₂ or HA.

The embodiment of FIG. 3B is advantageous, among other reasons, because no separate process is taken by the patient or the medical professional to start a subsequent chemical reaction. That is, the additional chemical reaction is started automatically after the coating dissolves.

FIG. 3C is a graph illustrating release of gas with sequential chemical reaction of the reactant substance, according to one embodiment. The graph includes two curves, a curve in broken line indicating a single injection of water or saline solution for the chemical reaction with the magnesium, and a curve in a solid line indicating multiple injections of water for sequential chemical reaction of the magnesium. As shown in FIG. 3C, the curve in broken line exhibit abrupt initial increase in the release of H₂ gas followed by slow plateauing over time. In contrast, the curve in solid line exhibit gradual initial climb of H₂ gas release, which is resumed after each additional injection of water or saline solution (indicated by “2^{nd}” and “3^{rd}” in FIG. 3C). Hence, by exposing the magnesium to multiple injections of water or saline solution, more gradual inflation of the tissue expander may be achieved.

Other mechanisms may be used to prevent abrupt initial inflation of the tissue expander. For example, different pieces of the first reactant substance may be mixed with different additional materials to control the rate of the chemical reaction.

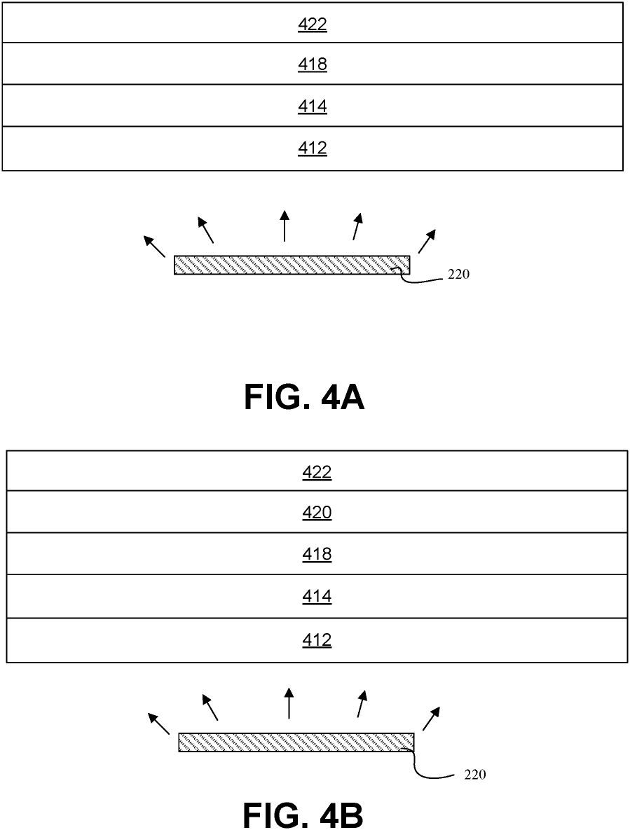

To render the film impermeable to gas while meeting certain functional requirements, the film that constitute the tissue expander may include multiple layers. FIG. 4A is a conceptual diagram illustrating a film of a tissue expander including four layers, according to one embodiment.

An innermost layer 412 of the film may be a polymer that does not react with the gas produced as a result of the chemical reaction while being sufficiently flexible to deform upon increase of the pressure of the gas. The polymer suitable for this purpose may include, but not limited to, polymer such as polyethylene, natural rubber, cellulose acetate, polysulfone (PSU), polyimide (PI), polyetherimide (PEI), nylon, polyurethane, tetrabromo polycarbonate, poly(vinyl trimethylsilane), polyvinyl fluoride (PVF), polyperfluorinated ethylene propylene (F46), polyvinylidene fluoride (PVDF), polyethylene (PE), and polyethylene terephthalate (PET).

A subsequent layer 414 may be a metal layer to prevent leaking of the gas. The layer 414 may be deposited on the outer surface of the innermost layer 412. The metal layer may be embodied using, for example, aluminum, titanium, tantalum, gold, platinum, silver or any combination thereof. Such metal layer may be formed on the innermost layer 412 using, for example, coating, wrapping or deposition methods such as atomic layer deposition, chemical vapor deposition, electroplating or thermal deposition.

Another layer 418 may be provided on the external surface of the metal layer 414 to protect the metal layer 414 from cracking or rupturing due to the inflation of the tissue expander. The layer 418 may be embodied using, for example, polymer such as polyethylene, natural rubber, cellulose acetate, polysulfone (PSU), polyimide (PI), polyetherimide (PEI), nylon, polyurethane, tetrabromo polycarbonate, poly(vinyl trimethylsilane), polyvinyl fluoride (PVF), polyperfluorinated ethylene propylene (F46), polyvinylidene fluoride (PVDF), and polyethylene terephthalate (PET).

The outermost layer 422 is a biocompatible material. The outermost layer 422 contacts patent’s tissues, and hence, is made of materials such polydimethylsiloxane (PDMS), poly(ethylene oxide), polyhydroxyethylmethacrylate (pHEMA), poly(methyl methacrylate (PMMA), polytetrafluoroethylene (PTFE), and polyamides (PA).

The film constituting the tissue expander may include additional materials. FIG. 4B is a conceptual diagram illustrating a film of a tissue expander including five layers, according to one embodiment. The structure of the film in FIG. 4B is the same as that of FIG. 4A except that an additional layer 420 is provided between layers 418, 422. The additional layer 420 may be a layer for providing strength to the film such as nylon.

The embodiments described above with references to FIGS. 4A and 4B are merely illustrative, and the film of the tissue expander may include fewer or more layers that these embodiments. For example, an extra layer may be added to provide antimicrobial effect. The extra layer may include, for example, silver.

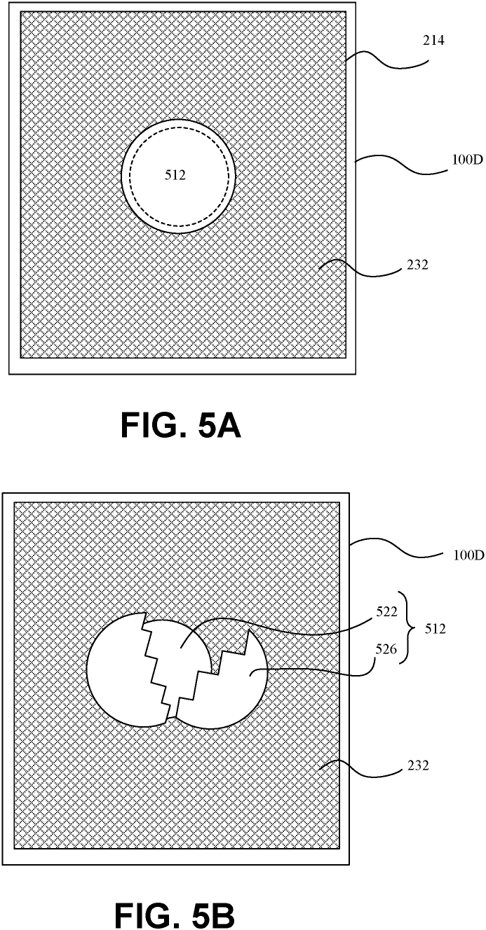

FIG. 5A is a plan view of tissue expander 100D with first reactant substance 522 enclosed in casing 526, according to one embodiment. Unlike the embodiments described above with reference to FIGS. 2A through 3B, the tissue expander 100D of FIG. 5A has an internal chamber 214 that is initially filled with second reactant substance 232 and an ampoule 512 immersed in the second reactant substance 232. The ampoule 512 includes the first reactant substance 522 and an outer casing 526 that separates the first reactant substance 522 from the second reactant substance 232.

As shown in FIG. 5B, the outer casing 526 of the ampoule 512 may be broken or ruptured to allow the first reactant substance 522 to come into contact with the second reaction substance 232, and thereby start the chemical reaction for producing the gas. The produced gas expands the internal chamber and inflates the tissue expander 1000D. The ampoule 512 may be broken or ruptured during a surgery when the tissue expander 1000D is implanted in a patient. A medical professional may twist or hammer the ampoule 512 so that the outer casing 526 breaks and exposes the first reactant substance 522 to the second reaction substance 232.

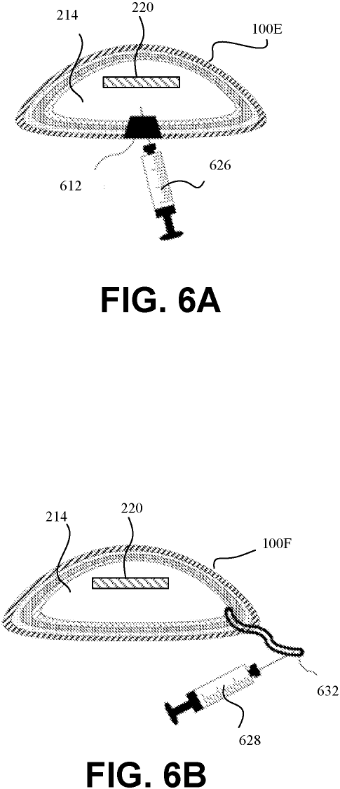

FIG. 6A is a conceptual diagram illustrating tissue expander 100E with septum 612, according to one embodiment. The tissue expander 100E has internal chamber 214 where first reactant substance 220 is placed. The tissue expander 100E includes septum 612 attached to an aperture of a film that constitutes the tissue expander 100E.

The tissue expander 100E does not include any storage chamber. Instead, a syringe 626 with a needle may be used to penetrate the septum 612 and inject the second reactant substance 232 in a liquid form. That is, the syringe 626 functions as an external source for providing the second reaction substance 232 to the tissue expander 100E. The injected second reactant substance starts a chemical reaction with the first reactant substance 220 that produces gas. After injecting the second reactant substance, the needle may be pulled out of the septum 612. The septum is made of a resilient material, and the hole formed by the needle is closed after removing the needle.

Although a needle is used to inject the second reactant substance, the inflation of the tissue expander 100E is achieved primarily through the gas produced after the injection of the second reactant substance and not by the volume of second reactant substance injected into the tissue expander 100E. Hence, the inflation of the tissue expander 100E occurs gradually and reduces or eliminates a patient’s pain due to the abrupt inflation of the tissue expander.

FIG. 6B is a conceptual diagram illustrating tissue expander 100F with a flexible conduit 632, according to one embodiment. The tissue expander 100F is similar to the tissue expander 100E but includes a flexible conduit 632 instead of a septum. The flexible conduit 632 may be embodied as a hose connected an internal chamber 214 of the tissue expander 100F, and is used to carrying the second reactant substance into the internal chamber 214 injected by syringe 628. That is, the syringe 628 functions as an external source for providing the second reaction substance 232 to the tissue expander 100F.

To prevent the gas from leaking out from the internal chamber 214 via the flexible conduit 632, the flexible conduit 632 may include a check valve or the outer opening of the flexible conduit 632 may be sealed off after injecting the second reactant substance. Alternatively, the entrance of the flexible conduit 632 may be provided with a self-sealing septum.

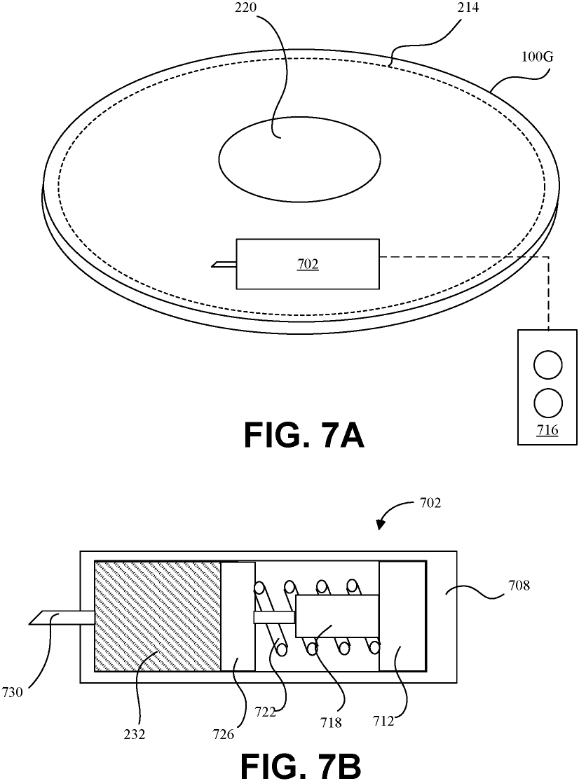

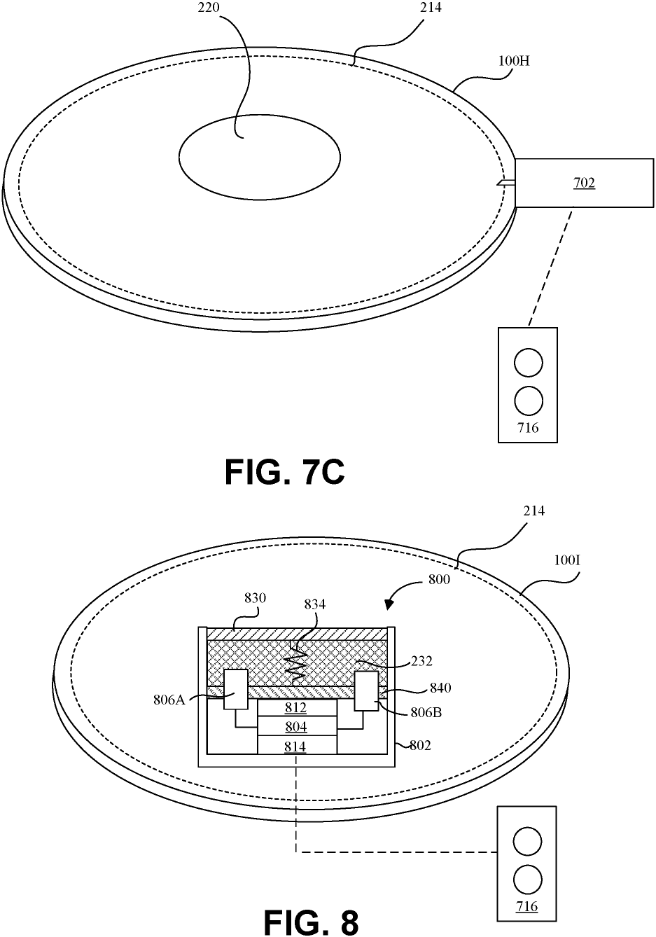

FIG. 7A is a perspective view of tissue expander 100G with a remotely controlled valve assembly 702, according to one embodiment. An internal chamber 214 of the tissue expander 100G includes first reactant substance 220 while the valve assembly 702 contains second reactant substance 232 that reacts with the first reactant substance 220 to produce gas. The valve assembly 702 is controlled wirelessly by a remote controller 716 to release controlled amount of the second reactant substance 232 into the internal chamber 214 so that an appropriate rate of chemical reaction occurs within the internal chamber 214. The remote controller 716 may be operated by the patient or a medical professional based on assessment of pain or discomfort associated with the inflation of the tissue expander 100G.

FIG. 7B is a cross-sectional diagram of the valve assembly 702 of FIG. 7A placed within the internal chamber 214 of the tissue expander 100G, according to one embodiment. The valve assembly 702 may include, among other components, an exit port 730, a plunger 726, a motor 718, a biasing spring 722, a control circuit 712, and a housing 708 enclosing at least a subset of these components. When a wireless signal is received from the remote controller 716, the control circuit 712 operates the motor 718 so that the plunger 726 moves toward the exit port 730. As a result, the second reactant substance 232 is discharged via the exit port 730. The biasing spring 722 provides biasing force to the plunger 726 so that the amount of force exerted by the motor 718 to move the plunger 726 may be reduced.

FIG. 7C is a perspective view of tissue expander 100H attached with a remotely controlled valve assembly 702, according to one embodiment. The tissue expander 100H is substantially identical to the tissue expander 100G of FIG. 7B except that the valve assembly 702 is placed outside the internal chamber 214. Rather, only the exit port 730 penetrates the film to provide the second reactive substance into the internal chamber 214.

FIG. 8 is a conceptual diagram of tissue expander 100I with electrolysis device 800, according to one embodiment. Instead of using the chemical reaction of two reactive substances, tissue expander 100I has only a single reactive substance that produces gas a result of electrolysis. The electrolysis device 800 generates current in its electrodes to perform the electrolysis on reactant substance 232 within the electrolysis device 800 in an internal chamber 214 of the tissue expander 100I.

The electrolysis device 800 may include, among other components, a power source 812, a wireless communication circuit 814, a driving circuit 804, electrodes 806A, 806B,a membrane 830, a sealing wall 840, and a housing 802 enclosing at least a subset of these components. The wireless communication circuit 814 communicates with a remote controller 716 to receive instructions to start or stop the electrolysis or control the voltage/current level associated with the electrolysis. The driving circuit 804 is connected to the power source 812 and provides current to the electrodes 806A, 806B that are exposed to the reactant substance 232 enclosed between the membrane 830 and the sealing wall 840. The amount or rate of gas to be produced by electrolysis may be controlled, for example, by adjusting a voltage difference across the electrodes 806A, 806B or by adjusting the duty cycle in a pulse-width modulation (PWM) scheme to turn on or off current to the electrodes 806A, 806B.

The membrane 830 may be made of material that is impermeable to liquid but permeable to gas. As a result, the reactant substance 232 in liquid form may not pass through the membrane 830 but gas generated by electrolysis passes through the membrane 830 into the internal chamber 214. As in previous embodiments, the gas released in the internal chamber 214 inflates the tissue expander 100I. By providing the membrane 830, the liquid form of the reactant substance 232 remains between the membrane 830 and the internal chamber 214 while the gas is released outside of electrolysis device 800 into the internal chamber 214. In this way, bubbles of the gas are prevented from forming within the reactant substance 232. Such bubbles may impede or interfere with the electrolysis process by blocking the contact of the electrodes 806A, 806B with the reactant substance 232. Further, a tension mechanism 834 such as a tension spring or an elastic band may be provided in or around the internal space of the electrolysis device 800 to shrink the space storing the reactant substance 232, thereby ensuring that the electrodes 806A, 806B maintain contact with the reactant substance 232 despite the reduced volume of the reactant substance 232 due to the electrolysis.

The reactive substance 232 for electrolysis may be water. When a water molecule is decomposed by electrolysis, oxygen and hydrogen gas are formed. Specifically, at a cathode (e.g., electrode 806A), hydrogen gas is produced according to the following reaction:

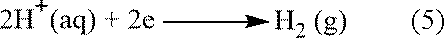

At an anode (e.g., electrode 806B),oxygen is produced as a result of the following reaction:

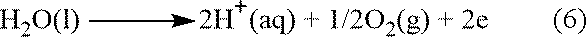

To enhance the rate of reaction, appropriate electrolyte such as NaCl may be added in the reactant substance 220 (e.g., water).

Various aqueous solution may be used as the reactant substance for producing gas using electrolysis. For example, aqueous solution with various types of salt or including substances such as HCl, HNO₃, KOH and NaOH may be used.

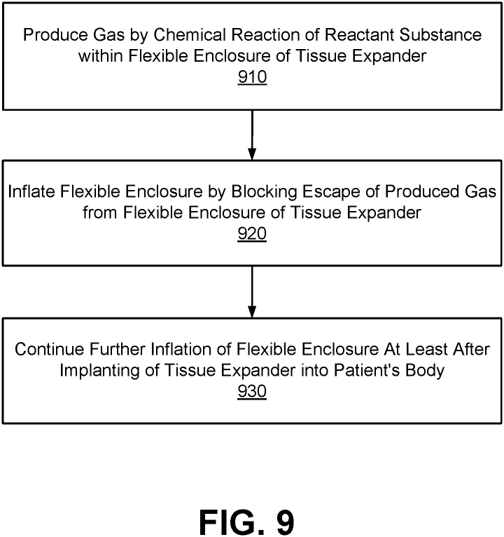

FIG. 9 is a flowchart illustrating a process of operating a tissue expander, according to one embodiment. Gas is produced 910 by a chemical reaction of reactant substance contained, as liquid or solid material, within a flexible enclosure of the tissue expander. The chemical reaction may occur as a result of the reactant substance coming into contact with another reactive substance. Alternatively, the chemical reaction may occur in the form of electrolysis of the reactant substance in the flexible enclosure.

The flexible enclosure is inflated 920 by blocking escape of the produced gas from the flexible enclosure. For this purpose, the flexible enclosure may be formed by a film that is impermeable to the gas. The film is also flexible to enable inflation of the tissue expander.

The inflation of the flexible enclosure is continued 930 at least after implanting the flexible enclosure into patient’s tissue. The gas continues to be produced after implanting, and hence, the flexible enclosure inflates not only during the time at which the tissue expander is implanted, but for a prolonged time after the tissue expander is implanted. The tissue expander may continue to expand over multiple days, weeks or even months after implanting.

The processes and their sequence as described above with reference to FIG. 9 are merely illustrative. Additional processes may be added or some of the processes may be performed in parallel. For example, the process of producing gas 910 may be performed in parallel with the inflating 920 of the flexible enclosure.

While particular embodiments and applications have been illustrated and described, it is to be understood that the invention is not limited to the precise construction and components disclosed herein and that various modifications, changes and variations which will be apparent to those skilled in the art may be made in the arrangement, operation and details of the method and apparatus disclosed herein without departing from the spirit and scope of the present disclosure.

1. A tissue expander comprising:
a flexible enclosure at least part of which is configured to contact and expand a patient’s surgical site, the flexible enclosure formed with an internal chamber and impermeable to gas;
a first reactant substance included within the internal chamber of the flexible enclosure as liquid or solid material; and
a second reactant substance in the flexible enclosure, the second reactant substance causing a chemical reaction to start by coming into contact with the first reactant substance, the chemical reaction starting at a location exposed to an interior wall of the internal chamber of the flexible enclosure, the chemical reaction continuing at the location at least after implanting of the flexible enclosure into the patient’s surgical site to produce the gas that inflates the flexible enclosure.

2. (canceled)

3. The tissue expander of claim 1, wherein the first reactant substance comprises a gas producing metal or sodium percarbonate and the second reactant substance comprises an aqueous solution.

4. The tissue expander of claim 3, wherein the gas producing metal is a metal selected from a group consisting of magnesium, molybdenum, tungsten and zinc.

5. The tissue expander of claim 1, wherein the flexible enclosure is formed with a storage chamber configured to enclose the second reactant substance, a passage between the internal chamber and the storage chamber, and a collapsible wall in the passage, and wherein the collapsible wall is configured to be ruptured by an external force to enable the second reactant substance to flow into the internal chamber.

6. The tissue expander of claim 5, wherein the flexible enclosure is formed with at least one additional storage chamber configured to enclose the second reactant substance, at least one additional passage between the internal chamber and the at least one additional storage chamber, and at least one additional wall configured to be ruptured by applying additional external force to provide additional second reactant substance to the internal chamber.

7. The tissue expander of claim 1, wherein the first reactant substance is coated with a coating material to expedite, delay or prolong the chemical reaction.

8. The tissue expander of claim 1, wherein the internal chamber is filled with the second reactant substance, and the first reactant substance is enclosed in an encapsulation that prevents the chemical reaction, and wherein the encapsulation is ruptured to expose the first reactant substance to the second reactant substance to initiate the chemical reaction.

9. The tissue expander of claim 1, further comprising a septum attached to the flexible enclosure and configured to be penetrated to provide the second reactant substance into the internal chamber to cause the chemical reaction, the septum further configured to be sealed after providing the second reactant substance into the internal chamber.

10. The tissue expander of claim 1, further comprising a flexible conduit extending into the internal chamber to carry the second reactant substance into the internal chamber to cause the chemical reaction.

11-23. (canceled)

24. The tissue expander of claim 1, wherein the first reactant substance is provided in different portions coated with a coating material of different thicknesses, the coating material dissolving after contacting the second reactant substance.

25. The tissue expander of claim 1, wherein the first reactant substance is provided in at least two portions, one of the at least two portions not coated with a coating material but others of the at least two portions coated with the coating material.

26. The tissue expander of claim 1, further comprising a wireless communication circuit configured to control the chemical reaction.

27. A tissue expander comprising:
a flexible enclosure at least part of which is configured to contact and expand a patient’s surgical site, the flexible enclosure formed with an internal chamber and impermeable to gas; and
an electrolysis device in the internal chamber and configured with internal space for storing reactant substance, and the electrolysis device comprising:
electrodes configured to perform electrolysis of the reactant substance to produce gas in the internal space, the electrolysis occurring in the internal space at least after implanting of the flexible enclosure into the patient’s surgical site,
a control circuit connected to the electrodes to provide current to the electrodes to perform the electrolysis, and
a membrane impermeable to the reactant substance but permeable to the gas to release the produced gas into the internal chamber and expand the flexible enclosure.

28. The tissue expander of claim 27, wherein the electrolysis device further comprises:
a wireless communication circuit configured to receive a wireless signal that instructs the control circuit to provide the current to the electrodes.
